(19) United States
(12) Patent Application Publication
(10) Pub. No.: US 2014/0150796 A1
(43) Pub. Date: Jun. 5, 2014

(54) SYSTEM AND METHOD FOR DETECTING MINIMAL VENTILATION SUPPORT WITH PROPORTIONAL ASSIST VENTILATION PLUS SOFTWARE AND REMOTE MONITORING

(71) Applicant: COVIDIEN LP, Mansfield, MA (US)

(72) Inventor: Gary Milne, Louisville, CO (US)

(73) Assignee: COVIDIEN LP, Mansfield, MA (US)

(21) Appl. No.: 13/690,648

(22) Filed: Nov. 30, 2012

(51) Int. Cl. A61M 16/00

(52) U.S. Cl. CPC A61M 16/0057
USPC 128/205.23

(57) ABSTRACT

Methods and systems are provided that determine whether a patient is receiving minimal support from a ventilator. In one embodiment, a ventilation system may determine a value of a peak inspiratory pressure (PIP) and a value of a positive end-expiratory pressure (PEEP) of a patient. The ventilation system may determine a pressure differential between the value of PIP and the value of PEEP and compare the pressure differential to a threshold. The ventilation system may determine that the patient is receiving minimal support if the pressure differential is below the threshold. Further, the ventilation system may provide an indication of the detection of minimal support.

### BACKGROUND

The present disclosure relates generally to medical devices, and more particularly, to medical devices that provide respiratory support to a patient, such as ventilators.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the course of treating a patient, a medical device may be used to control the flow of air, foods, fluids, or other substances to the patient. For example, ventilators may be used to provide supplemental oxygen support to the patient. Such ventilators typically include a source of pressurized oxygen, which may be delivered to the patient through a conduit. The ventilators may also monitor and display one or more breathing characteristics of the patient during ventilation. The ventilators may use the monitored one or more breathing characteristics to determine appropriate ventilation parameters for the patient. Additionally, a caregiver may evaluate the one or more breathing characteristics to adjust the ventilation parameters set by the ventilator.

In many instances, it may be desirable to wean the patient from the ventilator as soon as a caregiver determines that the patient may be able to support his or her ventilation and oxygenation. Thus, the caregiver may consider the breathing characteristics of the patient, as well as the level of support delivered to the patient by the ventilator, to assess respiratory function. In particular, the caregiver may wish to monitor the breathing characteristics of the patient and the level of ventilation support delivered to the patient to determine whether the patient is ready to perform a spontaneous breathing trial. A spontaneous breathing trial is a period of unassisted patient breathing or a period in which the patient receives minimal support from a ventilator operating in a support mode or an assist mode (i.e., the patient receives ventilation support for the patient's spontaneous breaths). The caregiver may monitor one or more physiological parameters of the patient to determine whether the patient is tolerating the spontaneous breathing trial or is exhibiting signs of failing the spontaneous breathing trial. The completion of a successful spontaneous breathing trial may indicate that the patient is ready to be liberated from the ventilator. However, the caregiver may not readily assess that the patient is receiving minimal support from the ventilator.

### BRIEF DESCRIPTION OF THE DRAWINGS

Advantages of the disclosed techniques may become apparent upon reading the following detailed description and upon reference to the drawings in which:

FIG. 1 is a block diagram of ventilation system including a ventilator in accordance with an embodiment;

FIG. 2 is a flow diagram of a method for providing an indication of minimal support detected using the ventilation system of FIG. 1 in accordance with an embodiment;

FIG. 3 is a flow diagram of a method for providing a recommendation for a spontaneous breathing trial using the ventilation system of FIG. 1 in accordance with an embodiment;

FIG. 4 is an illustration of a display of the ventilator of FIG. 1 including an indication of minimal support detected in accordance with an embodiment;

FIG. 5 is an illustration of a display of the ventilator of FIG. 1 including an indication of minimal support detected and a recommendation to increase a preset support percentage delivered by the ventilator of FIG. 1 in accordance with an embodiment; and

FIG. 6 is an illustration of a display of the ventilator of FIG. 1 including an indication of minimal support detected and a recommendation for a spontaneous breathing trial in accordance with an embodiment.

### DETAILED DESCRIPTION OF THE SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As noted above, a ventilator may monitor one or more breathing characteristics of a patient and use the breathing characteristics to set and adjust one or more ventilation parameters. For example, in a ventilator operating under proportional assist ventilation plus (PAV+) mode, a caregiver may set a percentage of support to be delivered to the patient, and the ventilator may automatically adjust the inspiratory pressure based on the patient's breathing characteristics and the patient's effort or flow demand. More specifically, the ventilator may deliver an inspiratory flow to the patient at a pressure to overcome the preset percentage of the patient's total work of breathing. For example, if the patient's lungs become more compliant and the total work of breathing decreases, the ventilator may reduce the pressure delivered to achieve the preset percentage of support. If the patient's flow demand increases (e.g., the patient begins to take larger and/or faster breaths) and the work of breathing increases, the ventilator may increase the pressure delivered to achieve the preset percentage of support. A higher pressure delivered to the patient (i.e., the pressure to ventilate) may indicate a higher level of ventilation support. Generally, when the pressure to ventilate drops below a threshold, the patient may be considered on minimal ventilation support. However, the caregiver may not readily assess when the patient is receiving minimal support.

Accordingly, the disclosed embodiments provide a system and method for providing an indication to a caregiver that a patient is receiving minimal support from a ventilator by providing a ventilation system that is configured to monitor the pressure differential between the peak inspiratory pressure (PIP or Ppeak) and the positive end-expiratory pressure (PEEP) of a patient. As defined herein, the pressure to ventilate is the difference between the PIP of the patient and the PEEP of the patient. Thus, the ventilation system may compare the pressure to ventilate to a predetermined threshold for minimal support and provide an indication when the pressure to ventilate is below the threshold. However, PIP and PEEP may fluctuate over time and thus, the pressure to ventilate may drop below the threshold momentarily. As such, the ventilation system may monitor the pressure to ventilate over a period of time and provide the indication of minimal support when the pressure to ventilate is below the threshold for minimal support for a predetermined percentage of the time. For example, a caregiver may set the threshold for minimal support to approximately seven centimeters of water (7 cm H₂O), a threshold time period to four hours, and a percentage threshold to approximately 70 percent. When the pressure to ventilate drops below approximately 7 cm H₂O, the ventilation system may begin collecting historical data of the pressure to ventilate. Once four hours of historical data is collected, the ventilation system may provide the indication of minimal support detected in response to determining that the pressure to ventilate was below approximately 7 cm H₂O for at least approximately 70 percent of the four hours. As will be described in more detail below, the values of the minimal support threshold, the threshold time period, and the percentage threshold may vary.

In certain embodiments, the ventilation system may additionally determine whether PIP and PEEP are below respective thresholds. In particular, a threshold, or a threshold range, may be determined for PIP and PEEP and may be individualized for the patient. Comparing PIP and PEEP to respective thresholds may be desirable for various circumstances. For example, the ventilation system may provide an alarm in response to determining that PIP and/or PEEP is above the respective threshold to alert the caregiver that the patient's condition is worsening and/or that the patient may benefit from an adjustment of one or more settings on the ventilator. Moreover, in circumstances in which the pressure to ventilate is below the threshold, but PIP and/or PEEP is above its respective threshold, it may be desirable to direct the caregiver's immediate attention to the parameter, rather than an indication of minimal support. Additionally or alternatively, the ventilation system may be configured to detect both conditions (i.e., minimal support and PIP and/or PEEP above the respective threshold) and to provide an alarm and a recommendation to the caregiver to increase the preset support percentage to increase the ventilation support delivered to the patient. That is, if the patient is receiving minimal support and PIP and/or PEEP is above its respective threshold, it may indicate that the patient may benefit from an increase in ventilation support.

Furthermore, in certain embodiments, the ventilation system may provide a recommendation that the patient may be ready to perform a spontaneous breathing trial. As noted above, a spontaneous breathing trial is a period of unassisted patient breathing or a period in which the patient receives minimal support from a ventilator operating in a support mode or an assist mode (i.e., the patient receives ventilation support for the patient's spontaneous breaths). The caregiver may monitor one or more physiological parameters of the patient during the spontaneous breathing trial to determine whether the patient is tolerating the spontaneous breathing trial. The completion of a successful spontaneous breathing trial may indicate that the patient is ready to be liberated from the ventilator. As described above, it may be desirable to liberate the patient from the ventilation system as soon as the caregiver determines that the patient may be able to support his or her ventilation and oxygenation. The indication of minimal ventilation support may be an indication that the patient is ready to be weaned from the ventilation system (i.e., is ready to perform a spontaneous breathing trial). However, it may be desirable to monitor one or more additional physiological parameters of the patient that may be related to the patient's readiness to wean. That is, to reduce the likelihood that the patient is taken off the ventilator too early, the ventilation system may compare additional physiological parameters to their respective thresholds or threshold ranges before providing the recommendation for a spontaneous breathing trial.

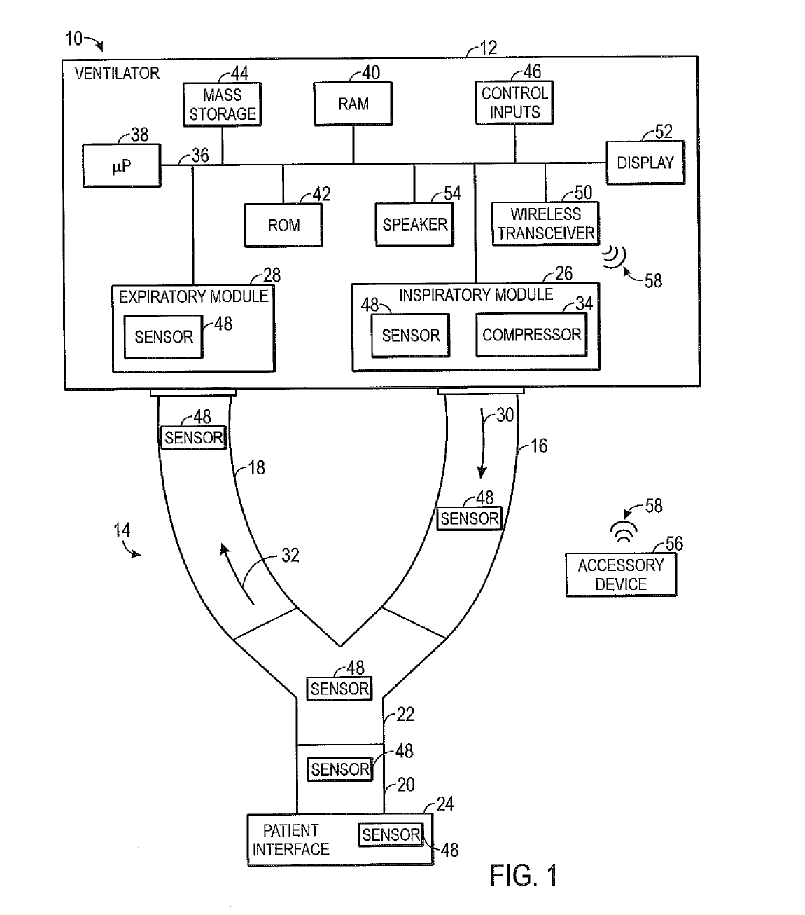

With the foregoing in mind, FIG. 1 illustrates a ventilation system 10 for providing respiratory support to a patient. The ventilation system 10 may include a ventilator 12 connected to a respiratory circuit 14. The respiratory circuit 14 may be in fluid communication with a source of respiratory gas and may enable one-way flow of inspired gases towards the patient and one-way flow of expired gases away from the patient. In particular, the respiratory circuit 14 may include an inspiratory conduit 16, an expiratory conduit 18, and a patient conduit 20. The inspiratory, expiratory, and patient conduits 16, 18, and 20, may be connected to one another by a Y-connector (i.e., a “wye” connector) 22, which may be connected to a patient interface 24. The patient interface 24 may be any suitable patient interface, such as an endrotracheal tube, a tracheotomy tube, or a breathing mask placed over the nose and/or mouth of the patient. Furthermore, the system 10 may include any number of connectors or medical tubing to provide the respiratory gas from the source to the lungs.

The ventilator 12 may include an inspiratory module 26 and an expiratory module 28 for circulating respiratory gases to and from the patient via the respiratory circuit 14 and the patient interface 24. Accordingly, the inspiratory module 26 may be coupled to the inspiratory conduit 16 for providing respiratory gases, represented by arrow 30, and the expiratory module 28 may be coupled to the expiratory conduit 18 for receiving respiratory gases, represented by arrow 32. As used herein, the respiratory gas may be air, oxygen, nitrogen, carbon dioxide, vaporized water, vaporized medicines, or any combination thereof. The inspiratory module 26 may be configured to receive a source of respiratory gas and to pressurize the respiratory gas via a compressor 34. Additionally or alternatively, the inspiratory module 26 may receive a source of pressurized respiratory gas, such as a compressed air wall outlet or a tank of pressurized respiratory gas. Furthermore, the inspiratory and expiratory modules 26 and 28 may include various suitable components, such as circuitry, valves, filters, tubing, and/or sensors. In one embodiment, the inspiratory and expiratory modules 26 and 28 may be coupled to an internal bus 36 and controlled by a processor 38 to regulate the pressure and/or flow of the respiratory gas delivered and removed. The processor 38 may be configured to control the operation of the inspiratory and expiratory modules 26 and 28 based at least in part upon a ventilator operating mode, such as an assist mode (e.g., PAV+). In a PAV+ mode, the inspiratory module 26 may be configured to deliver an inspiratory flow to the patient at a pressure selected to achieve a proportion (e.g., a preset support percentage) of the patient's total work of breathing.

The processor 38 may access and execute coded instructions, such as for implementing the algorithms discussed herein, from one or more storage components of the ventilator 12, such as a RAM 40, ROM 42, and/or a mass storage device 44. For example, code encoding executable algorithms may be stored in the RAM 40, the ROM 42, and/or the mass storage device 44 (such as a magnetic or solid state hard drive or memory or an optical disk or memory) and accessed and operated according to processor 38 instructions using stored data. In certain embodiments, the RAM 40, the ROM 42, and/or the mass storage device 44 may store information related to one or more settings of the ventilator 12, one or more coefficients or equations for calculating patient physiological parameters, and patient data. For example, patient data such as normal values or ranges of respiratory resistance and compliance for various patient populations may be stored. The processor 38 may also receive information related to ventilation settings and/or patient data from a caregiver via one or more control inputs 46. For example, a caregiver may input a patient's gender, age, weight, ideal body weight, and/or condition (e.g., asthma, emphysema, chronic obstructive pulmonary disease, acute respiratory distress syndrome, etc.) which may be used in the selection of the normal values of respiratory resistance and compliance, as well as alarm conditions. Additionally, the processor 38 may receive information from one or more sensors 48 of the ventilation system 10, as will be described in more detail below. In certain embodiments, the processor 38 may also receive information related to patient physiological parameters from other medical devices (e.g., a pulse oximeter or an electrocardiography device) via a wireless transceiver 50. The received information may be stored the RAM 40, the ROM 42, and/or the mass storage device 44 and may be used in calculations for determining one or more physiological parameters of a patient relating to respiratory function. The ventilator 12 may also include a display 52 and/or a speaker 54, which may be used to convey information about the calculated physiological parameters and/or ventilation parameters or settings to the caregiver. Furthermore, the wireless transceiver 50 may be configured to transmit information to one or more accessory devices 56 via wireless communication 58 to enable the caregiver to remotely monitor the calculated physiological parameters and/or the ventilation parameters. For example, the one or more accessory devices 56 may include a remote computer (e.g., located at a nurse's station), a pager, a smart phone, a smart watch, a laptop computer, a handheld computing device, or a cloud computing device.

As noted above, the processor 38 may calculate the one or more physiological parameters based in part upon signals received from the one or more sensors 48 in the ventilation system 10. The sensors 48 may obtain signals related to the flow and/or pressure of the supplied and returned respiratory gases, which may be indicative of the patient's respiratory function. In particular, for PAV+ mode, the sensors 48 may obtain signals representative of the patient's instantaneous inspiratory flow and lung volume. Accordingly, any suitable sensor 48 for determining flow, pressure, concentrations of components in the patient's respiratory gas, or any other desired parameter may be used. For example, the sensors 48 may be pressure sensors, flow sensors, or optical sensors. Additionally, the sensors 48 may generate signals related to certain physiological parameters, such as pressure and flow, which may be used by the processor 38 to derive other physiological parameters. For example, the processor 38 may be configured to derive PIP, PEEP, tidal volume, inspiratory time, expiratory time, respiratory rate, plateau pressure, alveolar pressure, inspiratory reserve volume, expiratory reserve volume, vital capacity, functional residual capacity, a ratio of inspiratory time to expiratory time (I:E), respiratory resistance, respiratory compliance, and/or any other physiological parameter. In particular, the processor 38 may be configured to integrate the determined instantaneous inspiratory flow to derive the lung volume. Additionally, the processor 38 may be configured to graphically represent one or more physiological parameters on the display 52 and to provide visual or audible indications related to one or more physiological parameters via the display 52 or the speaker 54, which will be described in more detail below.

The sensors 48 may be placed at any suitable location for measuring physiological parameters of the patient. For example, the ventilation system 10 may include sensors 48 located within the ventilator 12, such as within the inspiratory and the expiratory modules 26 and 28. Additionally, sensors 48 may be disposed about the respiratory circuit 14. In certain embodiments, inspiratory, expiratory, and patient conduits 16, 18, and 20 and the Y-connector 22 may each include one or more sensors 48. In particular, for PAV+ mode, the sensors 48, may be located about the Y-connector 22 to generate a signal representative of the Y-connector flow, to be used for the determination of instantaneous inspiratory flow. Additionally, it may be desirable to obtain measurements near the lungs and/or near the diaphragm of the patient. As such, one or more sensors 48 may be located within or disposed about the patient interface 24. In certain embodiments, to reduce the dead space volume within the respiratory circuit 14 and/or the patient interface 24, the sensors 48 may be embedded in the walls of the inspiratory, expiratory, and patient conduits 16, 18, and 20, the Y-connector 22, and/or the walls of the patient interface 24 (e.g., the walls of an endrotracheal or tracheotomy tube). Additionally, it should be noted that one or more lead wires (not shown) may couple the sensors 48 to the ventilator 12 to power the sensors 48 and transmit the signals.

As noted above, the processor 38 may be configured to cause the inspiratory and expiratory modules 26 and 28 to operate under a PAV+ mode. In certain embodiments, the ventilator 12 may include PAV+ software, which may allow the inspiratory and expiratory modules 26 and 28 to operate under the PAV+ mode. In PAV+ mode, the inspiratory module 26 is triggered to deliver an inspiratory flow to the patient by the patient's inspiratory muscles (i.e., initiation of a breath). After the patient ceases inspiration, the inspiratory module 26 also stops delivering the inspiratory flow. In this manner, the PAV+ mode may allow the patient to set his or her own breathing pattern. As noted above, the inspiratory module 26 may be configured to deliver the inspiratory flow at a specific pressure selected by the processor 38 to overcome a preset percentage of the patient's total work of breathing. For example, the caregiver may select or input the desired percentage of support using the control inputs 46. In certain embodiments, the support percentage may be between approximately 5 and 95 percent and may be adjusted in approximately 5 percent increments, or in any other desired increment. The patient's total work of breathing, and thus the selected pressure, may be based at least in part upon the patient's breathing characteristics, such as resistance, compliance, and flow demand (i.e., the patient's respiratory rate and volume). Because the patient's breathing characteristics may be initially unknown, the inspiratory module 26 may be configured to deliver one or more maneuver breaths to estimate the patient's resistance and compliance. Following the maneuver breaths, the inspiratory module 26 may deliver the inspiratory flow using the preset support percentage. The instantaneous inspiratory flow and volume may be monitored to adjust the pressure of the inspiratory flow to overcome the preset support percentage of the total work of breathing. The caregiver may input additional ventilator settings to adjust the parameters of the inspiratory flow. For example, the caregiver may select via the control inputs 46 a value of PEEP to increase the patient's oxygenation.

Because the pressure of the delivered inspiratory flow depends on the patient's resistance, compliance, and flow demand, the patient's PIP may also depend on the resistance, compliance, and flow demand. More specifically, an increase in resistance or a decrease in compliance may increase the work of breathing. Additionally, if the patient's flow demand increases, the work of breathing may also increase. Thus, the ventilator 12 may provide more ventilation support (i.e., an inspiratory flow at a higher pressure) to the patient to overcome the preset percentage of the increased work of breathing, which may increase the patient's PIP. Similarly, if the patient's resistance decreases, compliance increases, and/or flow demand decrease, the ventilator 12 may decrease the ventilation support (i.e., an inspiratory flow at a lower pressure), which may decrease the PIP of the patient. In this manner, the amount of support delivered to the patient may increase as the patient's effort (i.e., flow demand) increases and decrease as the patient's effort decreases. As such, if the patient experiences respiratory distress and/or fatigue, the patient's effort may decrease, causing the patient to receive a lower amount of total ventilation support.

As such, the amount of support delivered to the patient by the ventilator 12 may vary based at least in part upon the patient's resistance, compliance, flow demand, and work of breathing. However, as discussed above, the caregiver may not readily assess that the patient is receiving minimal ventilation support. Accordingly, as noted above, it may be desirable to monitor the pressure to ventilate the patient, because the pressure to ventilate may be an indicator of the ventilation support. As defined herein, the pressure to ventilate is the difference between the PIP of the patient and the PEEP of the patient. Moreover, it may be desirable to determine whether the pressure to ventilate is below a threshold for minimal support and to provide an indication to the caregiver when the pressure to ventilate is below the minimal support threshold. In this manner, the indication may alert the caregiver that the ventilator 12 may be providing minimal support to the patient.

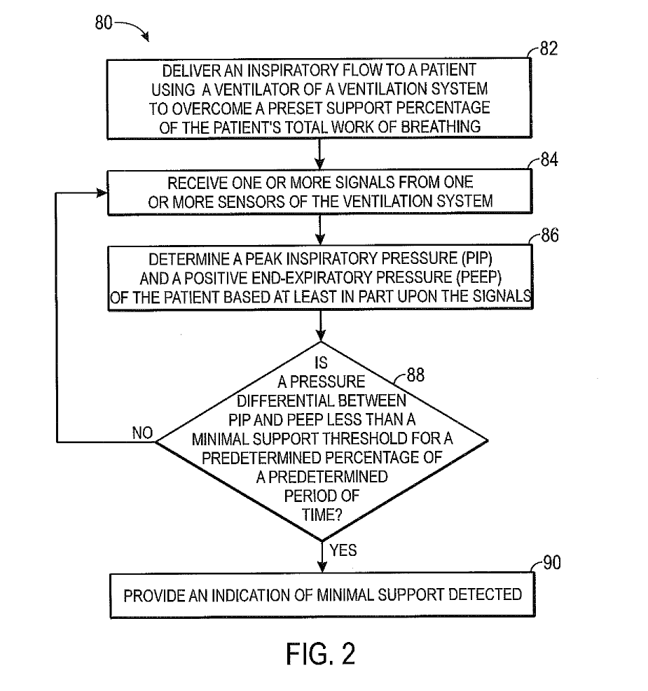

With the foregoing in mind, FIG. 2 illustrates a method 80 for monitoring the pressure to ventilate a patient in accordance with some embodiments. The method 80 may be performed as an automated procedure by a system, such as the ventilation system 10. In addition, certain steps of the method 80 may be performed by a processor, or a processor-based device such as the ventilator 12 that includes instructions for implementing certain steps of the method 80.

The method 80 may include delivering an inspiratory flow to a patient using a ventilator of a ventilation system (e.g., the ventilator 12 of the ventilation system 10) to overcome a preset support percentage of the patient's total work of breathing (block 82). As described above, the ventilator 12 may be configured to deliver the inspiratory flow at a specific pressure to overcome a proportion (i.e., the preset support percentage) of the patient's total work of breathing. The method 80 may also include receiving one or more signals from one or more sensors, such as the sensors 48, of the ventilation system 10 (block 84). For example, the processor 38 may receive the signals from the sensors 48 via one or more leads. The processor 38 may process and/or analyze the signals and may utilize information from the signals to determine the patient's instantaneous inspiratory flow and lung volume and to derive the patient's resistance and compliance. The processor 38 may then use these parameters to determine an appropriate delivered pressure to overcome the preset support percentage of the total work of breathing. Furthermore, the processor 38 may be configured to determine the PIP and the PEEP of the patient based at least in part on the one or more signals (block 86). In certain embodiments, the processor 38 may derive the PIP and the PEEP from one or more determined physiological parameters. For example, the processor 38 may determine the pressure and flow of the patient's respiratory system using the one or more signals and may derive PEEP and PIP from pressure and flow using known equations. In certain embodiments, the processor 38 may monitor PIP and PEEP continuously. For example, the processor 38 may calculate PIP and PEEP for each breath of the patient. In other embodiments, the processor 38 may be configured to calculate PIP and PEEP periodically. For example, the processor 38 may calculate PIP and PEEP for every few breaths of the patient.

To determine whether the ventilator 12 is providing minimal support to the patient, the processor 38 may calculate the difference between PIP and PEEP (i.e., the pressure to ventilate) and compare the difference to a predetermined threshold for minimal support. In certain embodiments, the threshold for minimal support may be approximately 8 cm H₂O, 7 cm H₂O, or 6 cm H₂O. The caregiver may input or select the desired minimal support threshold via the control inputs 46. In one embodiment, the minimal support threshold may be stored in the RAM 40, the ROM 42, and/or the mass storage device 44.

However, as noted above, the delivered pressure may vary over time based on the patient's resistance, compliance, and flow demand, and thus, the PIP of the patient may vary over time. As such, the pressure to ventilate may momentarily fall below the threshold for minimal support, but may not necessarily indicate that the patient is receiving minimal support. For example, the patient's flow demand may fluctuate as the patient transitions from asleep to alert. As such, it may be desirable to monitor the pressure to ventilate over a period of time to account for any fluctuations.

Accordingly, the method 80 may include determining whether the difference between PIP and PEEP (i.e., the pressure to ventilate) has been less than the minimal support threshold for a predetermined percentage of a predetermined period of time (block 88). In certain embodiments, this determination may include monitoring the pressure to ventilate and storing historical data for the pressure to ventilate once the pressure to ventilate falls below the minimal support threshold. The processor 38 may analyze the historical data to determine whether the criteria for minimal support have been met. For example, the processor 38 may determine whether the pressure to ventilate has been less than the minimal support threshold for at least approximately 60 percent, 70 percent, 80 percent, or 90 percent of the predetermined period of time. Additionally, the period of time may be between approximately 30 minutes and 240 minutes, 45 minutes and 180 minutes, 60 minutes and 120 minutes, or any other suitable period of time

In certain embodiments, two or more threshold combinations of the threshold percentage and the threshold period of time may be stored in the RAM 40, the ROM 42, and/or the mass storage device 44 and utilized by the processor 38. For example, it may be desirable to set a lower threshold period of time (e.g., 30 minutes) for a higher threshold percentage (e.g., at least approximately 90 percent). Additionally, it may be desirable to set a long threshold period of time (e.g., two hours) for a lower threshold percentage (e.g., at least approximately 70 percent). Thus, in certain embodiments, the processor 38 may be configured to monitor the pressure to ventilate with regard to the two or more threshold combinations. As such, if the first threshold combination (i.e., at least approximately 90 percent for 30 minutes) is not met, the processor 38 may then monitor the pressure to ventilate with regard to the second threshold combination (i.e., at least approximately 70 percent for two hours). It should be noted that any suitable threshold combination may be selected.

If the pressure to ventilate was not below the minimal support threshold for the predetermined percentage of the predetermined period of time, the processor 38 may continue monitoring the signals from the sensors 48 (block 84). In certain embodiments, the processor 38 may subsequently stop collecting historical data for the pressure to ventilate until the pressure to ventilate falls below the minimal support threshold again. In other embodiments, the processor 38 may be configured to analyze the stored historical data to determine whether a new starting point for the threshold period of time may be selected. That is, if a first portion of the threshold period of time had an average percentage of minimal support below the threshold percentage, but a second portion of the threshold period of time had an average percentage of minimal support above the threshold percentage, the processor 38 may be configured to restart the predetermined period of time at the beginning of the second portion.

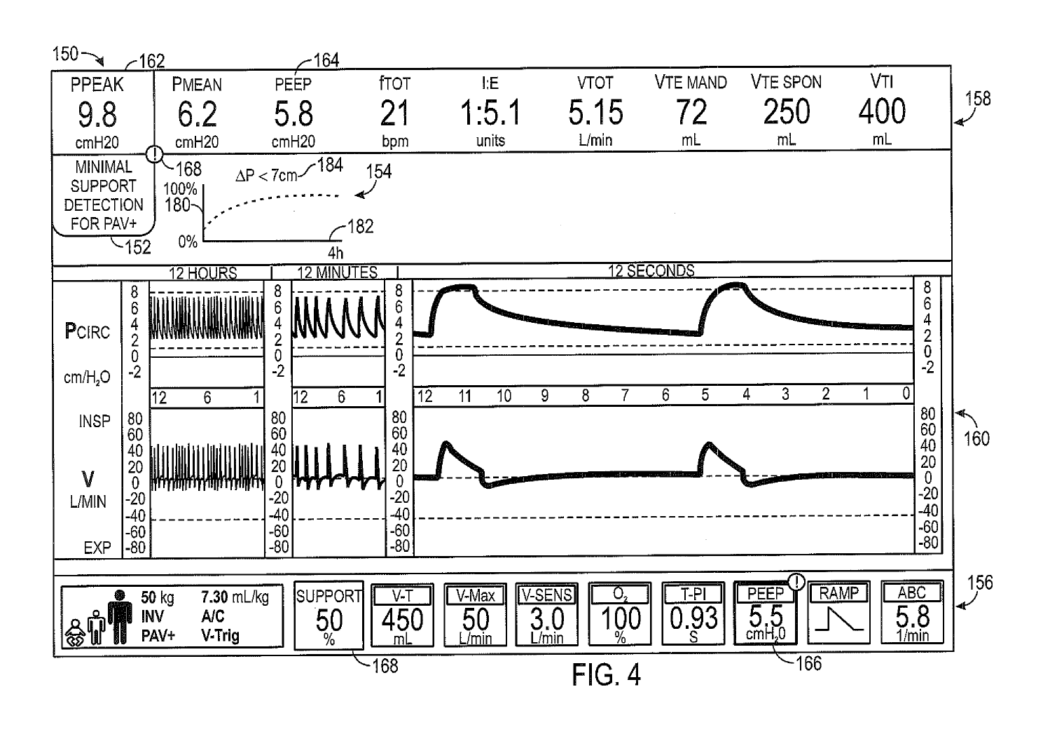

In response to determining that the pressure to ventilate was below the minimal support threshold for the predetermined percentage of the predetermined period of time, the processor 38 may provide an indication of minimal support detected (block 90). More specifically, the indication of minimal support detected may be an indication that the ventilator 12 is delivering minimal support to the patient. The indication of minimal support detected may be user-perceptible. For example, the processor 38 may cause the display 52 to display a visual indication, which may be textual, graphical, or any other suitable indication. Various embodiments of the indication of minimal support detected will be described in more detail below with respect to FIGS. 4-6. Additionally or alternatively, the processor 38 may cause the speaker 54 to provide an audible indication, such as an alarm or a beep. In this manner, the ventilator 12 may direct the caregiver's attention to the ventilator 12, so that the caregiver may be aware that the ventilator 12 is delivering minimal support to the patient. By providing the indication, the ventilator 12 may alert the caregiver that the patient may benefit from a reassessment of his or her condition and treatment. That is, a patient on minimal support may be ready to perform a spontaneous breathing trial to determine whether the patient is ready to be liberated from the ventilator 12, or if the patient has not improved sufficiently, the patient may benefit from an increase in ventilation support (i.e., an increase in the support percentage).

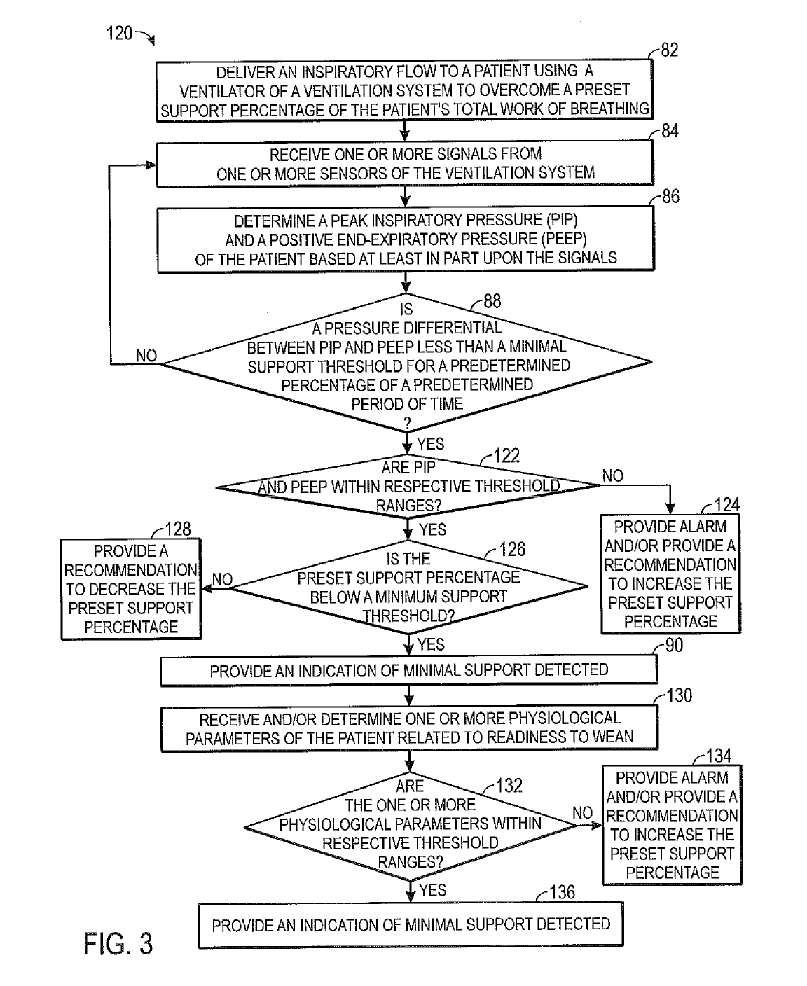

Because the indication of minimal support detected may indicate disparate potential conditions of the patient, it may be desirable to provide additional information to the caregiver. That is, the processor 38 may be configured to consider one or more physiological parameters, in addition to PIP and PEEP, to evaluate the patient's condition. With the foregoing in mind, FIG. 3 illustrates a method 120 for monitoring the pressure to ventilate a patient and providing a recommendation to a caregiver for a spontaneous breathing trial in accordance with some embodiments. The method 120 may be performed as an automated procedure by a system, such as the ventilation system 10. In addition, certain steps of the method 120 may be performed by a processor, or a processor-based device such as the ventilator 12 that includes instructions for implementing certain steps of the method 120.

Similar to the method 80, the method 120 may include delivering an inspiratory flow to a patient using a ventilator of a ventilation system (e.g., the ventilator 12 of the ventilation system 10) to overcome a preset support percentage of the patient's total work of breathing (block 82). Additionally, the method 120 may include receiving one or more signals from the one or more sensors 48 of the ventilation system 10 (block 84) and determining the PIP and PEEP of the patient based at least in part upon the one or more signals (block 86). Next, the method 120 may determine whether the difference between PIP and PEEP (i.e., the pressure to ventilate) is below the minimal support threshold for the predetermined percentage of the predetermined period of time (block 88).

If the processor 38 determines that the pressure to ventilate has been below the minimal support threshold for the predetermined percentage of the predetermined period of time, the processor 38 may determine whether PIP and PEEP are within respective threshold ranges (block 122). It may be desirable to compare PIP and PEEP to threshold ranges, because there may be various combinations of PIP and PEEP that result in a value of the pressure to ventilate that is below the minimal support threshold, but may not indicate that the patient is ready for a spontaneous breathing trial. For example, the patient's pressure to ventilate may be approximately 8 cm H₂O, but the patient's PIP and PEEP may be approximately 21 cm H₂O and approximately 13 cm H₂O, respectively. These values may indicate that the patient has a decreased compliance or increased resistance and/or that the patient is struggling with oxygenation. Indeed, the caregiver may increase the value of PEEP, which generally is set between approximately 3 and 5 cm H₂O, to improve the patient's oxygenation. In one embodiment, a maximum threshold of the threshold range for PEEP may be approximately 8 cm H₂O. Additionally, if the patient is experiencing respiratory distress or fatigue, the patient's flow demand may decrease. As such, the patient may not be able to sustain a sufficient inspiratory time to receive a sufficient amount of ventilation support, because the inspiratory module 26 may be configured to deliver ventilation support synchronized with the patient's breathing. Thus, the patient's PIP may fall below a minimum threshold of the threshold range.

It should be noted, however, that the processor 38 may compare PIP and PEEP to their respective threshold ranges before calculating the pressure to ventilate and/or before monitoring the pressure to ventilate for the predetermined period of time. That is, in certain embodiments, it may be desirable to provide an alarm or indication if PIP and/or PEEP is not within the respective threshold range prior to, or instead of, monitoring the pressure to ventilate over the predetermined period of time. For example, if the patient experiences sleep apnea, the patient may periodically cease breathing. As noted above, the inspiratory module 26 operating under PAV+ mode may be configured to deliver an inspiratory flow synchronized with a spontaneous breath of the patient. Thus, if the patient momentarily ceases breathing, the ventilator 12 will also momentarily cease delivering ventilation support, and PIP may be below a minimal threshold of the threshold range. Accordingly, in certain situations, such as a patient with sleep apnea, it may be desirable to compare PIP and PEEP to their respective threshold ranges before calculating the pressure to ventilate and/or before monitoring the pressure to ventilate for the predetermined period of time to provide an earlier alarm to the caregiver regarding the patient's condition.

Moreover, in certain embodiments, comparing PIP and PEEP to threshold ranges may include comparing several values of PIP and PEEP stored in the historical data over the predetermined period of time to the their respective threshold ranges. For example, values of PIP and PEEP may be selected at predetermined intervals of the period of time or all of the values may be compared. Alternatively, an average of the values of PIP and PEEP over the predetermined period of time may be calculated and compared to the threshold ranges. This may be advantageous to identify any trends or reoccurring outliers in the values of PIP and PEEP, which may be indicative of a condition of the patient that may be causing the patient to be on minimal support. For example, in the case in which the patient has sleep apnea, by examining several values of PIP, the processor 38 may determine that the value of PIP decreases below the minimum threshold of the threshold range several times throughout the predetermined period of time or that an average value of PIP is below a minimum threshold of the threshold range.

Thus, if PIP or PEEP is outside of the respective threshold range, the method 120 may include providing an alarm and/or providing a recommendation to increase the preset support percentage to provide more ventilation support (block 124). It should be appreciated that the recommendation to increase the support percentage merely functions to direct the caregiver's attention to a possible solution that may remedy the physiological parameters that are out of range, and the caregiver may evaluate the patient to determine if increasing the support percentage may be beneficial.

If the processor 38 determines that both PIP and PEEP are below the respective threshold, the processor 38 may determine whether the preset support percentage is below a minimum support threshold (block 126). If the preset support percentage is above the minimum support threshold, the method 120 may include providing a recommendation to decrease the preset support percentage (block 128). That is, the patient may be tolerating the minimal support (i.e., the patient's PIP and PEEP are within their respective ranges), but the preset support percentage may be relatively high (e.g., over approximately 50 percent). As such, the patient may not be ready to perform a spontaneous breathing trial. Accordingly, it may be desirable to decrease the support percentage in increments (e.g., approximately five percent) and monitor how the patient tolerates the lower preset support percentage. The minimal support threshold may be any suitable threshold between approximately 5 percent and 95 percent and may be selected by the caregiver using the control inputs 46. In certain embodiments, the minimum support threshold may be between approximately 20 percent and 70 percent, 25 percent and 65 percent, 30 percent and 60 percent, 35 percent and 55 percent, or 40 percent to 50 percent. The processor 38 may cause the display 52 to display the recommendation, which may be textual or graphical.

If the preset support threshold is below the minimum support threshold, the processor 38 may provide an indication of minimal support detected (block 90). Furthermore, the processor 38 may be configured to provide the indication of minimal support detected in addition to providing the alarm and/or recommendation to increase the preset support percentage if PIP and/or PEEP is outside its respective threshold range. Alternatively, the processor 38 may provide the indication of minimal support detected in addition to the recommendation to decrease the preset support percentage if PIP and PEEP are within their respective threshold ranges and the preset support percentage is above the minimal support threshold. As described above, the processor 38 may cause the display 52 to display a visual indication, which may be textual, graphical, or any other suitable indication.

Next, the method 120 may include receiving and/or determining one or more physiological parameters of the patient related to the patient's readiness to wean (block 130). As noted above, while a pressure to ventilate below the minimal support threshold may indicate that the patient is ready to perform a spontaneous breathing trial, it may be advantageous to consider other physiological parameters related to readiness to wean to reduce the possibility that the patient is liberated from the ventilator 12 too early. In certain embodiments, the physiological parameters related to readiness to wean may include partial pressure of oxygen in the blood, fractional inspired oxygen, pH of arterial blood, compliance, resistance, and/or any other suitable physiological parameters. Additionally, since the patient effectively sets his or her breathing pattern while receiving PAV+ support, it may also be desirable to consider the patient's respiratory rate and tidal volume, as these parameters may be indicative of respiratory distress. Accordingly, the processor 38 may be configured to calculate or derive additional physiological parameters based at least in part upon the signals received from the sensors 48 of the ventilation system 10. Additionally or alternatively, the processor 38 may be configured to receive signals from other medical devices, such as a pulse oximeter and/or an end-tidal carbon dioxide (EtCo₂) monitor, via the wireless transceiver 50 or any other suitable means. Additionally, the caregiver may input values of physiological parameters via the control inputs 46.

The method 120 may include determining whether the one or more physiological parameters are within respective threshold ranges (block 132). Accordingly, the processor 38 may be configured to receive the threshold ranges from other medical devices via the wireless transceiver 50 or from the caregiver via the control inputs 46. In certain embodiments, the threshold ranges may be stored in the RAM 40, the ROM 42, and/or the mass storage device 44. In one embodiment, the ventilator 12 may be configured to receive data about the patient via the control inputs 46, such as age, gender, weight, and/or condition, and the processor 38 may be configured to select the appropriate threshold range from the RAM 40, the ROM 42, and/or the mass storage device 44, based on the inputted patient data.

If one or more of the physiological parameters are outside of their respective threshold ranges, the method 120 may include providing an alarm and/or providing a recommendation to increase the preset support percentage to provide more ventilation support (block 134). For example, if the processor 38 determines that the ventilator 12 is providing minimal support to the patient, but the patient's partial pressure of oxygen in the blood is less than a maximum threshold (e.g., approximately 60 mmHg) and/or the patient's oxygen saturation is less than a minimum threshold (e.g., approximately 90 percent), the patient may benefit from an increase in ventilation support to achieve adequate arterial oxygenation.

If the one or more physiological parameters are within their respective ranges, the method 120 may include providing a recommendation for a spontaneous breathing trial (block 136). That is, if the patient is receiving minimal ventilation support and the physiological parameters of the patient are within normal ranges, the patient may be ready to perform a spontaneous breathing trial to determine if he or she is ready to be liberated from the ventilator 12. It should be appreciated that the recommendation merely functions to direct the caregiver's attention to the patient's possible readiness to perform a spontaneous breathing trial, and the caregiver may evaluate the patient to determine if the patient may subjected to the spontaneous breathing trial. Furthermore, in certain embodiments, the recommendation for a spontaneous breathing trial may also include a recommendation to check patient vitals prior to the spontaneous breathing trial.

As noted above, the display 52 may display various visual indications of minimal support detected when the processor 38 determines that the patient is receiving minimal ventilation support, as well as recommendations that may be beneficial to the patient on minimal support. Additionally, as noted above, the ventilator 12 may be configured to transmit data via the wireless transceiver 50. Thus, the various visual indications of minimal support detected may be displayed on the display 52 and/or a display of the accessory device 56 (e.g., a remote computer or a smart phone). Accordingly, while the embodiments described below in FIGS. 4-6 are described in the context of the display 52, it should be noted that the embodiments may be displayed on any suitable display, which may be external to the ventilator 12. For example, FIG. 4 is an illustration 150 of the display 52 including an indication of minimal support detected 152, or minimal support detection, and a graph 154 of the stored historical data for the determination of minimal support detected. Additionally, the display 52 may display ventilator settings 156, calculated and/or derived physiological characteristics 158, and one or more graphs 160 relating to the calculated pressure and/or flow of the patient's respiratory circuit. As illustrated, the calculated and/or derived physiological characteristics 158 may include a value of the patient's PIP (i.e., P_{PEAK}) 162 and a value of the patient's PEEP 164. It should be noted that the ventilator settings 156 also include a value of PEEP 166, and PEEP 164 and PEEP 166 may be the same or different values. Additionally, the ventilator settings 156 may include a support percentage 168 (e.g., the preset support percentage). In certain embodiments, the support percentage 168 may range from approximately 5 percent to 95 percent and may be adjusted in 5 percent increments. Furthermore, certain ventilators 12 may be configured to operate in more than one operating mode, such as PAV+ mode, a volume support mode, a volume control mode, etc. As such, the display 52 may display various ventilator settings 156 that may not be set by the caregiver for particular modes. For example, in PAV+ mode, the caregiver may not set a target tidal volume 170.

The indication of minimal support detected 152 may be illustrated on the display 52 in any suitable means for conveying the indication to the caregiver. In certain embodiments, the indication of minimal support detected 152 may be a textual indication. The textual indication may specify the particular mode of the ventilator 12 (e.g., PAV+). As illustrated, the indication of minimal support detected 152 may be located below the value of PIP 162. Alternatively, the indication of minimal support detected 152 may be located below the value of PEEP 164, near an alarm display, below any of the calculated and/or derived physiological characteristics 158, or any other suitable location. Additionally, the indication of minimal support detected 152 may be displayed as a tab, a banner, a dialog box, or any other suitable type of display. The indication of minimal support detected 152 may also include a symbol 168, such as an exclamation point, an asterisk, a star, a stop sign, or any other symbol. In certain embodiments, the indication of minimal support detected 152 may be displayed in the same font and color as the value of PIP 162, the value of PEEP 164, and/or the value of PEEP 166. Furthermore, to assist the caregiver in identifying the variables involved, the processor 38 may be configured to alter the font, color, shading, and/or size of the indication of minimal support detected 152 and the values of PIP 162, PEEP 164, and PEEP 166 from the other elements on the display 52. Moreover, the indication of minimal support detected 152 and the values of PIP 162, PEEP 164, and PEEP 166 may be displayed with the same font, color, shading, and/or size (e.g., a larger size than the other calculated and/or derived physiological characteristics 158).

As noted above, the display 52 may also display the graph 154 of the stored historical data for the determination of minimal support detected. In particular, the graph 154 may illustrate the percentage of time that the pressure to ventilate is below the minimal support threshold (ordinate 180) against time (abscissa 182). The graph 154 may provide information to the caregiver regarding the fluctuations of the pressure to ventilate over the predetermined period of time. The graph 154 may also display the minimal support threshold, which may be illustrated as a title 184 of the graph 154. The graph 154 may be displayed below, above, or adjacent to the indication of minimal support detected 152, or in any other suitable location. The graph 154 may also be displayed in the same font, color, and/or shading of the indication of minimal support detected 152. Furthermore, the graph 154 may be initially displayed as a small element on the display 52 and may be enlarged and/or displaced when selected by the caregiver (e.g., via the control inputs 46 or by touching a touch-screen display 52).

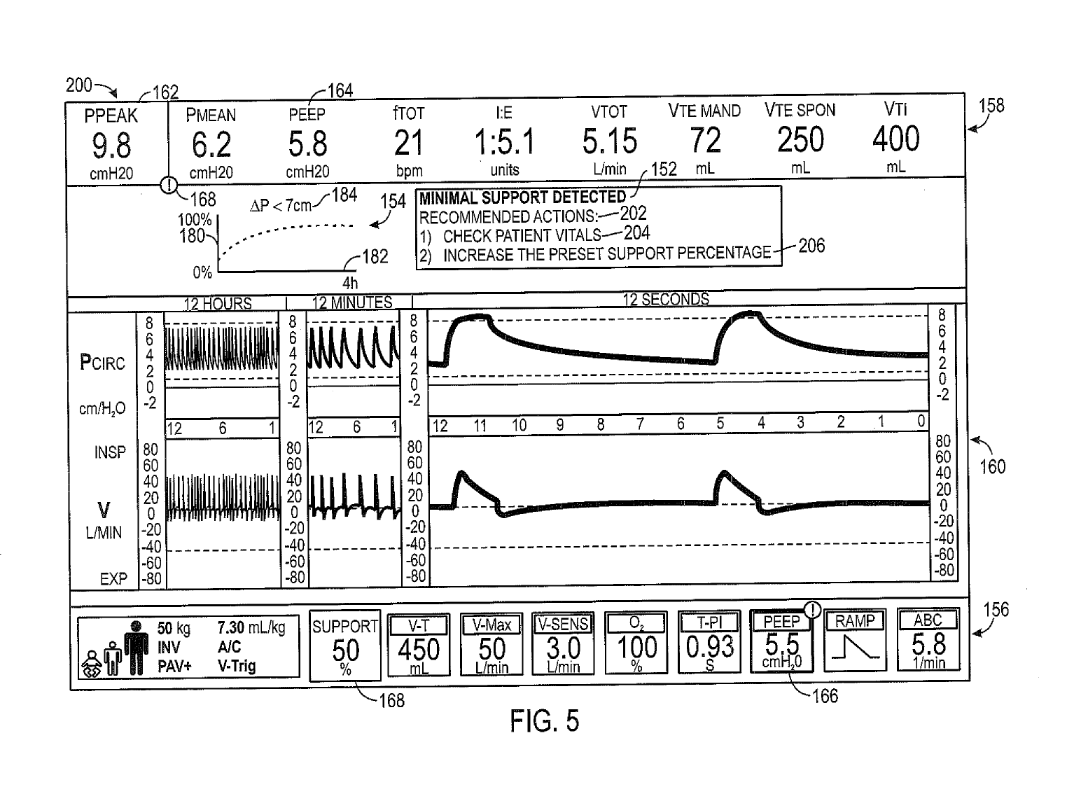

If the processor 38 determines that the patient is receiving minimal support and the value of PIP 158, the value of PEEP 160, or one of the physiological characteristics related to readiness to wean are outside of their respective thresholds, the processor 38 may cause the display 52 to display a recommendation to increase the ventilation support along with the indication of minimal support detected. For example, FIG. 5 is an illustration 200 of the display 52 including the indication of minimal support detected 152, the graph 154, and a recommendation 202. As illustrated, the recommendation 202 may be displayed in a dialog box along with the indication of minimal support detected 152, although other suitable means for display are contemplated. The recommendation 202 may include a list of suggested actions for the caregiver. For example, the recommendation 202 may include a recommendation to check the patient's vitals 204. The recommendation to check the patient's vitals 204 may alert the caregiver that at least one physiological parameter of the patient is outside of its respective threshold range. In certain embodiments, the recommendation to check the patient's vitals 204 may identify (e.g., textually display) the one or more physiological parameters outside of the threshold range. Additionally, the recommendation 202 may include a recommendation to increase the preset support percentage 206 to provide more ventilation support to the patient. In certain embodiments, the processor 38 may determine a suggested increase in the preset support percentage, based at least in part upon the calculated pressure to ventilate and the signals received from the sensors 48 of the ventilation system 10. Thus, the recommendation to increase the preset support percentage 206 may also include the suggested increase in the preset support percentage. In addition to providing the recommendation to increase the preset support percentage 206, the display 52 may also alter the font, color, shading, and/or size of the support percentage 158.

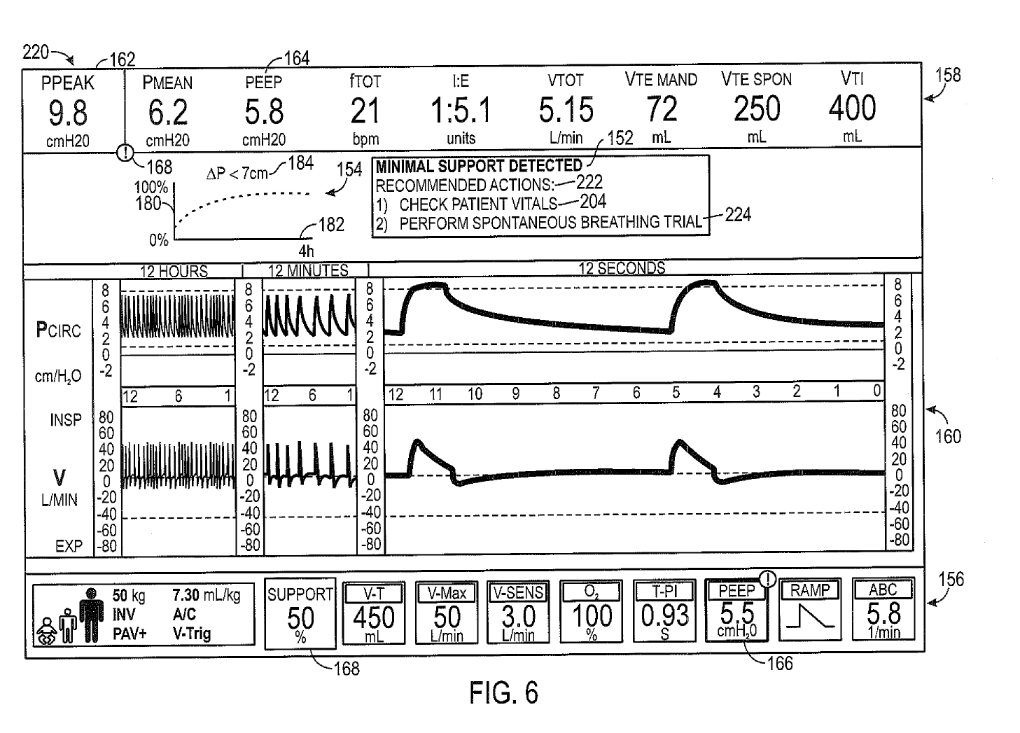

Alternatively, if the processor 38 determines that the patient is receiving minimal support and the value of PIP 158, the value of PEEP 160, and the physiological characteristics related to readiness to wean are within their respective thresholds, the processor 38 may cause the display 52 to display a recommendation for a spontaneous breathing trial with the indication of minimal support detected. For example, FIG. 6 is an illustration 220 of the display 52 including the indication of minimal support detected 152, the graph 154, and a recommendation 222. The recommendation 222 may include a recommendation 224 to have the patient perform a spontaneous breathing trial and/or to consider removing (i.e., liberating) the patient from the ventilator 12. That is, if the patient is receiving minimal support and the patient's physiological parameters are within respective normal ranges, the patient may have demonstrated the capacity to support his or her own breathing. Thus, following an assessment from the caregiver, the patient may be removed from the ventilator 12 without performing a spontaneous breathing trial. The recommendation 222 may also include the recommendation to check patient vitals 204, which may be displayed in a list format before the recommendation 224 for the spontaneous breathing trial. In this manner, the recommendation 222 may alert the caregiver that it may be beneficial to examine the patient and the ventilation system 12 to assess whether the patient may be ready for the spontaneous breathing trial and/or for liberation from the ventilator 12.

The disclosed embodiments may be interfaced to and controlled by a computer readable storage medium having stored thereon a computer program. The computer readable storage medium may include a plurality of components such as one or more of electronic components, hardware components, and/or computer software components. These components may include one or more computer readable storage media that generally store instructions such as software, firmware and/or assembly language for performing one or more portions of one or more implementations or embodiments of an algorithm as discussed herein. These computer readable storage media are generally non-transitory and/or tangible. Examples of such a computer readable storage medium include a recordable data storage medium of a computer and/or storage device. The computer readable storage media may employ, for example, one or more of a magnetic, electrical, optical, biological, and/or atomic data storage medium. Further, such media may take the form of for example, floppy disks, magnetic tapes, CD-ROMs, DVD-ROMs, hard disk drives, and/or solid-state or electronic memory. Other forms of non-transitory and/or tangible computer readable storage media not list may be employed with the disclosed embodiments.

A number of such components can be combined or divided in an implementation of a system. Further, such components may include a set and/or series of computer instructions written in or implemented with any of a number of programming languages, as will be appreciated by those skilled in the art. In addition, other forms of computer readable media such as a carrier wave may be employed to embody a computer data signal representing a sequence of instructions that when executed by one or more computers causes the one or more computers to perform one or more portions of one or more implementations or embodiments of a sequence.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A method, comprising:
receiving a signal from at least one sensor of a ventilation system, wherein the ventilation system is configured to deliver an inspiratory flow to a patient to overcome a present percentage of a total work of breathing of the patient;
determining a value of peak inspiratory pressure (PIP) and a value of positive end-expiratory pressure (PEEP) for the patient based at least in part upon the signal;
determining a pressure to ventilate of the patient, wherein the pressure to ventilate is a difference between the values of PIP and PEEP;
determining whether the pressure to ventilate is below a threshold for minimal support of the patient; and
providing an indication of minimal support detected in response to determining that the pressure to ventilate is below the threshold for minimal support.

2. The method of claim 1, wherein providing the indication of minimal support comprises providing a visual indication.

3. The method of claim 1, wherein determining whether the pressure to ventilate is below the threshold for minimal support comprises determining whether the pressure to ventilate is below the threshold for minimal support for a predetermined percentage of a predetermined period of time.

4. The method of claim 3, comprising displaying a graph illustrating a percentage of time that the pressure to ventilate is below the threshold for minimal support against time.

5. The method of claim 1, comprising determining whether the values of PIP and PEEP are both within respective threshold ranges.

6. The method of claim 5, comprising determining whether the preset percentage is below a minimum support threshold.

7. The method of claim 6, comprising providing a recommendation for the patient to perform a spontaneous breathing trial in response to determining that the values of PIP and PEEP are both within their respective threshold ranges and that the preset percentage is below the minimum support threshold.

8. The method of claim 6, comprising providing a recommendation to increase the preset percentage of the total work of breathing in response to determining that the value of PIP or the value of PEEP is outside its respective threshold range.

9. A ventilation system, comprising:
one or more sensors configured to generate one or more signals representative of a respiratory function of a patient;
a ventilator configured to receive the one or more signals from the one or more sensors and to be operatively coupled to the patient, wherein the ventilator comprises:
an inspiratory module configured to deliver an inspiratory flow to the patient to overcome a preset percentage of a total work of breathing of the patient;
a processor configured to:
determine a value of peak inspiratory pressure (PIP) and a value of positive end-expiratory pressure (PEEP) for the patient based at least in part upon the one or more signals;
determine whether a pressure to ventilate is below a threshold for minimal support of the patient, wherein the pressure to ventilate is a difference between the value of PIP and the value of PEEP; and
a display configured to display an indication of minimal support detected when the processor determines that the value of the pressure to ventilate is below the threshold for minimal support.

10. The ventilation system of claim 9, wherein the display is configured to display the indication of minimal support detected and the value of PIP or the value of PEEP with the same color, shading, or size.

11. The ventilation system of claim 9, wherein the display is configured to display the indication of minimal support detected when the processor determines that the value of the pressure to ventilate below the threshold for minimal support for a predetermined percentage of a predetermined period of time.

12. The ventilation system of claim 11, wherein the display is configured to display a graph illustrating a percentage of time that the value of the pressure to ventilate is below the threshold for minimal support against time.

13. The ventilation system of claim 11, wherein the processor is configured to compare the values of both PIP and PEEP to respective threshold ranges, and wherein the display is configured to display a recommendation to a user based at least in part upon the comparison.

14. The ventilation system of claim 12, wherein the recommendation comprises a recommendation to deliver a spontaneous breathing trial to the patient if the processor determines that the values of both PIP and PEEP are within their respective threshold ranges or a recommendation to increase the preset percentage of the total work of breathing if the processor determines that the value of PIP or the value of PEEP is outside its respective threshold.

15. The ventilation system of claim 9, wherein the threshold for minimal support is approximately seven centimeters of water (7 cm H₂O).

16. A ventilator, comprising:
an inspiratory module configured to deliver an inspiratory flow to a patient to overcome a preset percentage of a total work of breathing of the patient;
a processor configured to calculate a pressure to ventilate value of a patient and to determine whether the pressure to ventilate value is below a minimal support threshold for a predetermined percentage of a predetermined period of time; and
a display configured to display an indication of minimal support detected when the processor determines that the pressure to ventilate value is below the minimal support threshold for the predetermined percentage of the predetermined period of time.

17. The ventilator of claim 16, wherein the indication of minimal support detected comprises a list of suggested actions to a user.

18. The ventilator of claim 17, wherein the processor is configured to compare one or more physiological parameters of the patient to respective threshold ranges and to cause the display to display a recommendation to a user based at least in part upon the comparisons.

19. The ventilator of claim 18, wherein the recommendation comprises a recommendation to increase the preset percentage of the total work of breathing.

20. The ventilator of claim 18, wherein the recommendation comprises a recommendation to deliver a spontaneous breathing trial to the patient.
